(19) United States
(12) Patent Application Publication
(10) Pub. No.: US 2017/0065509 A1
(43) Pub. Date: Mar. 9, 2017

(54) FAT OR OIL EXTRACT OF BLACK GINGER AND METHOD FOR PRODUCING SAME

(71) Applicant: SUNTORY HOLDINGS LIMITED, Osaka (JP)

(72) Inventor: Naoki KASAJIMA, Osaka (JP)

(21) Appl. No.: 15/308,867

(22) PCT Filed: May 1, 2015
(86) PCT No.: PCT/JP2015/063089

§ 371 Date: Nov. 4, 2016

(30) Foreign Application Priority Data
May 9, 2014 (JP) 2014-097937

(51) Int. Cl. A61K 8/49; A23L 33/105; B01D 11/02; A23D 9/007

(52) U.S. Cl. CPC A61K 8/498; A23D 9/007; A23L 33/105; B01D 11/0288; A23V 2002/00

(57) ABSTRACT

There are provided black ginger extracts that contain reasonable amounts of methoxyflavones and which are reduced in undesirable flavors such as bitterness or in the intensity of a black purple color. To this end, oil or fat extracts are obtained from black ginger.

### TECHNICAL FIELD

The present invention relates to oil or fat extracts obtained from black ginger, as well as processes for their production, and so forth.

### BACKGROUND ART

Black ginger (Kaempferia parviflora) is a plant belonging to the Zingiberaceae family and is also known as black turmeric in Japan. Black ginger grows naturally in Southeast Asia and other regions, and in Thailand, it is a traditional herbaceous plant that is also known as Kra chai dahm.

Previous studies have revealed that principal components of black ginger are polyphenols including anthocyanidins and methoxyflavonoids, and gingerol and shogaol which are contained in Zingiber officinale Roscoe as well as curcumin contained in turmeric are absent from black ginger. Known to have a variety of efficacies including an anti-obesity action, an anti-ED action and a blood stream improving action, black ginger also finds extensive use in Japan as a supplement or as an ingredient of beverages. The component that is responsible for those efficacies is presumably methoxyflavones. Accordingly, isolated or purified methoxyflavones might be administered to obtain a variety of efficacies. However, such forms of methoxyflavones are expensive and in actual cases they are often replaced by black ginger extracts containing methoxyflavones.

As disclosed in Patent Document 1, etc., black ginger extracts containing methoxyflavones have heretofore been produced, mostly by extraction with hot water or hydrous alcohol. However, the extracts obtained by this method have distinctive flavors such as bitterness and present a black purple color. To solve the flavor problem, Patent Document 1 makes use of sugars, sugar alcohols, edible acids or artificial sweeteners.

### CITATION LIST

### Patent Literature

Patent Document 1: JP 2013-192513 A

### SUMMARY OF INVENTION

### Technical Problem

The conventional black ginger extracts have distinctive flavors such as bitterness and present a black purple color. These properties can be a problem when those extracts are used in foods and beverages, pharmaceuticals, cosmetics, and so on.

In this respect, the flavor and color at issue could become less noticeable if the yield of extraction were reduced. But then the concentrations of methoxyflavones as the active component would also be reduced.

The present invention has as its objective providing black ginger extracts that contain reasonable amounts of methoxyflavones and which are reduced in undesirable flavors such as bitterness or in the intensity of a black purple color.

### Solution to Problem

The present inventor conducted intensive studies to attain this objective and found that in oil or fat extracts obtained from black ginger, the black purple color which was peculiar to the conventional black ginger extracts was less noticeable or reduced in intensity. What is more, such oil or fat extracts were found to contain methoxyflavones adequately. The present invention has been accomplished on the basis of these findings.

Briefly, the present invention relates to, but is not limited to, the following.

1. An oil or fat extract, as obtained from black ginger, which comprises one or more members selected from the following eleven methoxyflavones, i.e., 5,7,3′,4′-tetramethoxyflavone, 3,5,7,3′,4′-pentamethoxyflavone, 5,7-dimethoxyflavone, 5,7,4′-trimethoxyflavone, 3,5,7-trimethoxyflavone, 3,5,7,4′-tetramethoxyflavone, 5-hydroxy-3,7,3′,4′-tetramethoxyflavone, 5-hydroxy-7-methoxyflavone, 5-hydroxy-7,4′-dimethoxyflavone, 5-hydroxy-3,7-dimethoxyflavone, and 5-hydroxy-3,7,4′-trimethoxyflavone, wherein a solution which is prepared from said extract and in which the total content of said eleven methoxyflavones is 5.0 mg/ml shows an absorbance of 0.10 or less as measured at a wavelength of 660 nm.
2. The extract as recited in 1 above, wherein the oil or fat is at least one member selected from among middle-chain fatty acid triglycerides, diacylglycerol, sesame salad oil, olive oil, soybean oil, rapeseed oil, corn oil, rice germ oil, sunflower seed oil, Perilla frustescens var. crispa oil, and Perilla frustescens var. frustescens oil.
3. A food or beverage comprising the extract as recited 1 or 2 above.
4. A cosmetic comprising the extract as recited 1 or 2 above.
5. A process for producing from black ginger an oil or fat extract which comprises one or more members selected from the following eleven methoxyflavones, i.e., 5,7,3′,4′-tetramethoxyflavone, 3,5,7,3′,4′-pentamethoxyflavone, 5,7-dimethoxyflavone, 5,7,4′-trimethoxyflavone, 3,5,7-trimethoxyflavone, 3,5,7,4′-tetramethoxyflavone, 5-hydroxy-3,7,3′,4′-tetramethoxyflavone, 5-hydroxy-7-methoxyflavone, 5-hydroxy-7,4′-dimethoxyflavone, 5-hydroxy-3,7-dimethoxyflavone, and 5-hydroxy-3,7,4′-trimethoxyflavone, which process comprises contacting a plant body of black ginger with an oil or fat and extracting at least one or more of said methoxyflavones.
6. The process as recited in 5 above, wherein the oil or fat is at least one member selected from among middle-chain fatty acid triglycerides, diacylglycerol, sesame salad oil, olive oil, soybean oil, rapeseed oil, corn oil, rice germ oil, sunflower seed oil, Perilla frustescens var. crispa oil, and Perilla frustescens var. frustescens oil.
7. The process as recited in 5 or 6 above, wherein the extraction is performed at 50 to 180° C.
8. A process for producing from black ginger an oil or fat extract which comprises one or more members selected from the following eleven methoxyflavones, i.e., 5,7,3′,4′-tetramethoxyflavone, 3,5,7,3′,4′-pentamethoxyflavone, 5,7-dimethoxyflavone, 5,7,4′-trimethoxyflavone, 3,5,7-trimethoxyflavone, 3,5,7,4′-tetramethoxyflavone, 5-hydroxy-3,7,3′,4′-tetramethoxyflavone, 5-hydroxy-7-methoxyflavone, 5-hydroxy-7,4′-dimethoxyflavone, 5-hydroxy-3,7-dimethoxyflavone, and 5-hydroxy-3,7,4′-trimethoxyflavone, which process comprises:

contacting a plant body of black ginger with water, a hydrophilic solvent or a mixture thereof and extracting one or more of said methoxyflavones; and

bringing an intermediate extract as obtained by said extraction into contact with an oil or fat and extracting said one or more methoxyflavones.

9. The process as recited in 8 above which further includes the step of evaporating the water, hydrophilic solvent or mixture thereof from the intermediate extract before the intermediate extract is contacted with the oil or fat and/or while they are kept in contact with each other.
10. The process as recited in 8 or 9 above, wherein the oil or fat is at least one member selected from among middle-chain fatty acid triglycerides, diacylglycerol, sesame salad oil, olive oil, soybean oil, rapeseed oil, corn oil, rice germ oil, sunflower seed oil, Perilla frustescens var. crispa oil, and Perilla frustescens var. frustescens oil.
11. The process as recited in any one of 5 to 10 above which further includes the steps of:

bringing an oil or fat containing extract as obtained by the step of contact with an oil or fat into contact with water, a hydrophilic solvent or a mixture thereof and extracting one or more of said methoxyflavones; and

subjecting to liquid-liquid separation a two-phase mixture as obtained during said extraction of said one or more methoxyflavones, whereby an extract comprising the water, hydrophilic solvent or mixture thereof is obtained that has been separated in the liquid-liquid separation step.

12. The process as recited in 11 above which further includes the step of removing the water, hydrophilic solvent or mixture thereof from the extract as obtained through the liquid-liquid separation step.
13. The process as recited in any one of 8 to 12 above, wherein the hydrophilic solvent is a C₁₋₃ alcohol and/or acetone.
14. An oil or fat extract as obtained by the process recited in any one of 5 to 13 above, which comprises one or more methoxyflavones selected from among said eleven methoxyflavones.

### Advantageous Effects of Invention

In the oil or fat extracts of the present invention, methoxyflavones as the active component have been selectively extracted, so the extracts contain reasonable amounts of methoxyflavones whereas the intensity of the undesirable color that has been presented by the conventional black ginger extracts is reduced. Since components that are difficult to extract with oil or fat might also contribute to the bitterness of black ginger, it can safely be concluded that bitterness is inevitably reduced in the oil or fat extracts of the present invention. In the case where such extracts contain residual oil or fat, they may be subsequently removed, also enabling the production of extracts (notably in a powder state) that are less colored and which have high methoxyflavone contents. Hence, the oil or fat extracts of the present invention provide greater ease for utilization in foods and beverages, pharmaceuticals, and cosmetics than the conventional black ginger extracts.

As a further advantage, the processes of the present invention for producing the foregoing oil or fat extracts do not use any special equipment, so the desired oil or fat extracts can be provided not only in an easy and inexpensive manner but also in large quantities.

Hence, the present invention will make great contribution to the utilization of methoxyflavones.

### BRIEF DESCRIPTION OF DRAWINGS

FIG. 1 is a photograph showing how an oil or fat extract of black ginger as prepared in accordance with the present invention and a hydrous alcohol extract of black ginger appear externally.

### DESCRIPTION OF EMBODIMENTS

(Oil or Fat Extracts)

The oil or fat extracts of the present invention are extracts as obtained from black ginger through extraction with oil or fat. The extracts contain methoxyflavones and are reduced in the intensity of a black purple color. The extracts may further contain oils or fats, especially those used in the extraction step.

The oil or fat extracts of the present invention are considered to differ in terms of the kinds of components contained, their proportions, etc. from extracts that are not derived from black ginger or from extracts that start from black ginger but which have not passed through extraction with oil or fat. For instance, extracts obtained from plants other than black ginger may contain methoxyflavones but their kinds and proportions would differ from those in the extracts of the present invention. Even if black ginger is used as the starting material, the proportions of methoxyflavones in extracts that are obtained from it by methods other than extraction with oil or fat, say, extraction with a hydrous alcohol are different from the values in the oil or fat extracts of the present invention. For further details of this point, refer to the Examples that will be described later in the subject specification. As for the extraction to be performed with oil or fat in the present invention, it may be applied directly to black ginger or indirectly, for example, to a liquid extract as obtained from black ginger using a solvent other than oil or fat, say, water, a hydrophilic solvent or a mixture thereof.

(Black Ginger)

Black ginger (Kaempferia parviflora) is a plant belonging to the Zingiberaceae family and easily available since it grows naturally or is cultivated in Southeast Asia and other regions.

For extraction, any part of black ginger may be employed. Exemplary parts that may be used include leaves, flowers, rhizomes, etc. and among these, rhizomes are preferred. While the plant body or part of black ginger may be subjected as such to extraction, they are preferably dried before being subjected to extraction. If desired, the dried plant body or part thereof may be subjected to solvent extraction either as such or after being ground to particles. The drying may be either sun-drying or machine drying. In the present invention, only one of the various states of black ginger described above may be employed or two or more of those states may be combined.

(Methoxyflavones)

The oil or fat extracts of the present invention contain one or more methoxyflavones. The term “methoxyflavones” as used herein means flavones having one or more methoxy groups. Methoxyflavones are typically selected from among compounds having the structure represented by the following formula (I):

### [Formula 1]

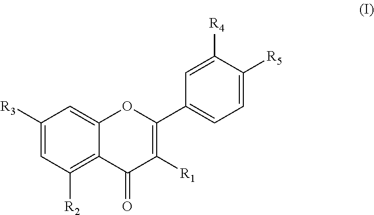

(where R₁, R₂, R₃, R₄ and R₅ are each independently hydrogen, a hydroxyl or a methoxy group, with at least one of R₁ to R₅ being methoxy.)

Preferably, the compound of formula (I) is selected from those identified in the following Table, i.e., 5,7,3′,4′-tetramethoxyflavone, 3,5,7,3′,4′-pentamethoxyflavone, 5,7-dimethoxyflavone, 5,7,4′-trimethoxyflavone, 3,5,7-trimethoxyflavone, 3,5,7,4′-tetramethoxyflavone, 5-hydroxy-3,7,3′,4′-tetramethoxyflavone, 5-hydroxy-7-methoxyflavone, 5-hydroxy-7,4′-dimethoxyflavone, 5-hydroxy-3,7-dimethoxyflavone, and 5-hydroxy-3,7,4′-trimethoxyflavone.

TABLE 1

| | R₁ | R₂ | R₃ | R₄ | R₅ | Compound Name |
|---|---|---|---|---|---|---|
| 1 | H | OCH₃ | OCH₃ | OCH₃ | OCH₃ | 5,7,3′,4′-tetramethoxyflavone |
| 2 | OCH₃ | OCH₃ | OCH₃ | OCH₃ | OCH₃ | 3,5,7,3′,4′-pentamethoxyflavone |
| 3 | H | OCH₃ | OCH₃ | H | H | 5,7-dimethoxyflavone |
| 4 | H | OCH₃ | OCH₃ | H | OCH₃ | 5,7,4′-trimethoxyflavone |
| 5 | OCH₃ | OCH₃ | OCH₃ | H | H | 3,5,7-trimethoxyflavone |
| 6 | OCH₃ | OCH₃ | OCH₃ | H | OCH₃ | 3,5,7,4′-tetramethoxyflavone |
| 7 | OCH₃ | OH | OCH₃ | OCH₃ | OCH₃ | 5-hydroxy-3,7,3′,4′-tetramethoxyflavone |
| 8 | H | OH | OCH₃ | H | H | 5-hydroxy-7-methoxyflavone |
| 9 | H | OH | OCH₃ | H | OCH₃ | 5-hydroxy-7,4′-dimethoxyflavone |
| 10 | OCH₃ | OH | OCH₃ | H | H | 5-hydroxy-3,7-dimethoxyflavone |
| 11 | OCH₃ | OH | OCH₃ | H | OCH₃ | 5-hydroxy-3,7,4′-trimethoxyflavone |

The oil or fat extracts of the present invention preferably contain at least one of compounds 1 to 11 identified in Table above. The oil or fat extracts more preferably contain at least two, more preferably at least three, more preferably at least four, more preferably at least five, more preferably at least six, more preferably at least seven, more preferably at least eight, more preferably at least nine, more preferably at least ten, and even more preferably eleven of compounds 1 to 11. For instance, the oil or fat extracts of the present invention preferably contain all of compounds 1 to 6.

In a preferred mode of the oil or fat extracts of the present invention, said at least one methoxyflavone is selected from group A consisting of 5,7,3′,4′-tetramethoxyflavone, 3,5,7,3′,4′-pentamethoxyflavone, 5,7-dimethoxyflavone, and 5,7,4′-trimethoxyflavone. Such oil or fat extracts which contain methoxyflavones of group A may also contain other compounds, say, at least one methoxyflavone that is selected from group B consisting of 3,5,7-trimethoxyflavone, 3,5,7,4′-tetramethoxyflavone, 5-hydroxy-3,7,3′,4′-tetramethoxyflavone, 5-hydroxy-7-methoxyflavone, 5-hydroxy-7,4′-dimethoxyflavone, 5-hydroxy-3,7-dimethoxyflavone, and 5-hydroxy-3,7,4′-trimethoxyflavone that are derived from black ginger.

The total content of methoxyflavones of group A as relative to the total content of methoxyflavones of groups A and B, which is expressed as A/(A+B), is preferably in excess of 0.65, more preferably 0.66 or greater, more preferably 0.67 or greater, more preferably 0.68 or greater, more preferably 0.69 or greater, more preferably 0.70 or greater, and more preferably 0.71 or greater on a molar basis (or weight basis). The value of A/(A+B) has no upper limit and may, for example, be 1.00 or smaller, 0.90 or smaller, or 0.80 or smaller. The present inventors have confirmed that the methoxyflavones of group A show a greater NOX inhibiting action than the methoxyflavones of group B. And Examples 9 and 10 of the present specification show that oil or fat extracts according to the present invention that have greater values of A/(A+B) exhibit a greater NOX inhibiting action than hydrophilic solvent extracts having smaller values of A/(A+B).

In the case where the oil or fat extracts of the present invention contain oil or fat, examples of the total content of the above-described eleven methoxyflavones in the extracts are 10 w/v % and below, preferably 0.1 to 10 w/v %, more preferably 0.1 to 5 w/v %, and even more preferably 0.1 to 2.5 w/v %. If, on the other hand, the oil or fat extracts of the present invention contain no oil or fat, examples of the total content of said eleven methoxyflavones in the extracts are 10 to 90 w/v %, preferably 20 to 70 w/v %, and more preferably 30 to 50 w/v %.

(Oils or Fats)

The oils or fats that can be employed to produce the oil or fat extracts of the present invention and which may be contained in such extracts are not particularly limited as long as they are capable of dissolving methoxyflavones. Typically, such oils or fats are at least one member selected from among middle-chain fatty acid triglycerides, diacylglycerol, sesame salad oil, olive oil, soybean oil, rapeseed oil, corn oil, rice germ oil, sunflower seed oil, Perilla frustescens var. crispa oil, and Perillafrustescens var. frustescens oil.

The term “middle-chain fatty acid” as used in connection with the middle-chain fatty acid triglycerides means fatty acids having 8 to 12 carbon atoms. At least one, preferably two, more preferably three of the fatty acid moieties that constitute said triglycerides are middle-chain fatty acids.

In the case where the oil or fat extracts contain oils or fats, the amount of the oils or fats that is present in the extracts is not particularly limited but typically is about 50 to about 100 w/w %.

(Color of Oil or Fat Extracts)

The oil or fat extracts of the present invention are reduced in the intensity of a black purple color. This can be effectively confirmed by measuring the light absorbance of the extracts.

Specifically, a solution of the extract is prepared in which the total content of the eleven methoxyflavones, i.e., 5,7,3′,4′-tetramethoxyflavone, 3,5,7,3′,4′-pentamethoxyflavone, 5,7-dimethoxyflavone, 5,7,4′-trimethoxyflavone, 3,5,7-trimethoxyflavone, 3,5,7,4′-tetramethoxyflavone, 5-hydroxy-3,7,3′,4′-tetramethoxyflavone, 5-hydroxy-7-methoxyflavone, 5-hydroxy-7,4′-dimethoxyflavone, 5-hydroxy-3,7-dimethoxyflavone, and 5-hydroxy-3,7,4′-trimethoxyflavone, is 5.0 mg/ml and the solution is measured for its absorbance at a wavelength of 660 nm. The thus measured absorbance is 0.10 or below in the present invention. The absorbance is preferably 0.07 or below, more preferably 0.05 or below. It should be emphasized here that the extracts (and the solution) need not contain all of the eleven methoxyflavones mentioned above. If, for example, the oil or fat extracts contain only nine of those eleven methoxyflavones, one may prepare a solution in which the total content of those nine methoxyflavones is 5.0 mg/ml and then measure its absorbance. In addition, unless otherwise specified, the term “absorbance” as used herein means a value for the case where the cell length (optical path length) is 10 mm. If the cell length of the device used for measurement is not 10 mm, the measured value of absorbance is converted to the value for the case of cell length=10 mm. It is also necessary to use a suitable blank for absorbance measurement.

In the present invention, the color intensity of substances obtained by extraction with oil or fat, especially, that of non-solvent substances is important. Hence, to prepare a solution to be subjected to absorbance measurement, only the amount of the solvent is adjusted and the amounts of other substances are not changed. In other words, no extrinsic substances other than the solvent may be added and none of the substances extracted may be removed. For example, if the oil or fat extracts contain no oil or fat, they are dissolved in a solvent, say, oil or fat to prepare a solution in which the total content of the eleven methoxyflavones is 5.0 mg/ml. If, on the other hand, the extracts contain an oil or fat, the amount of the solvent inclusive of the oil or fat is adjusted. If, for example, the total content of the eleven methoxyflavones in the extracts is greater than 5.0 mg/ml, a solvent such as an oil or fat is additionally supplied. If the extract contains an oil or fat and the total content of the eleven methoxyflavones in it is already 5.0 mg/ml, there is no need to adjust the amount of solvents, nor it is necessary to prepare a separate solution for absorbance measurement. It should, however, be noted that providing such an extract as a solution for absorbance measurement is also included, for convenience sake, in the case of “preparing a solution in which the total content of the eleven methoxyflavones is 5.0 mg/ml” in the present invention.

In general, light absorbance is measured in the absence of any insoluble matter. In the present invention, too, the absorbance of the extracts is measured in the state of a solution. Accordingly, if components precipitate from an extract in which the total content of the eleven methoxyflavones is 5.0 mg/ml, it is difficult to measure the absorbance of the extract directly. In this case, the extract is diluted with an oil or fat and the like to make a solution which is thereafter subjected to absorbance measurement; the measured absorbance is converted to the value for the case where the total content of the methoxyflavones in the extract is 5.0 mg/ml, whereupon the desired value or a nearby value is obtained. Even in the case where the total content of the methoxyflavones in the extract is less than 5.0 mg/ml even without effecting dilution, a measured absorbance may be converted in the manner just described above, whereupon the desired value or a nearby value is obtained.

(Other Components)

The oil or fat extracts of the present invention may, in addition to methoxyflavones and oil or fat, contain other components. For example, not only physiologically active components including vitamins such as vitamin E, minerals, hormones and nutrients but also stabilizers, antioxidants and other additives that are incorporated in formulating procedures may also be contained.

(Production Methods)

In another aspect, the present invention relates to processes for producing oil or fat extracts from black ginger.

Such processes, for example, comprise contacting a plant body of black ginger with an oil or fat and extracting one or more members selected from the following eleven methoxyflavones, i.e., 5,7,3′,4′-tetramethoxyflavone, 3,5,7,3′,4′-pentamethoxyflavone, 5,7-dimethoxyflavone, 5,7,4′-trimethoxyflavone, 3,5,7-trimethoxyflavone, 3,5,7,4′-tetramethoxyflavone, 5-hydroxy-3,7,3′,4′-tetramethoxyflavone, 5-hydroxy-7-methoxyflavone, 5-hydroxy-7,4′-dimethoxyflavone, 5-hydroxy-3,7-dimethoxyflavone, and 5-hydroxy-3,7,4′-trimethoxyflavone. A typical example of this method is described below.

First, a plant body of black ginger is provided. This plant body or part of it is dried and ground depending on the need. Subsequently, the plant body or its part is brought into contact with an oil or fat and subjected to extraction. The conditions for extraction are not particularly limited as long as they are capable of extracting methoxyflavones. Typical extraction temperatures are 50 to 180° C., 70 to 170° C., 70 to 150° C., 100 to 150° C., or 120 to 150° C. The extraction time is typically one minute to a day, 10 minutes to 10 hours, or 15 minutes to 5 hours. And the volume of the oil or fat to be used is typically 0.1 to 30 times or 0.5 to 15 times the weight of black ginger. Examples of the oil or fat to be used are as already described above.

Although not wishing to be bound by any theory, the present inventor assumes that in the process of this extraction, methoxyflavones transfer into the oil or fat whereas the components that are responsible for the black purple color of black ginger will remain in the plant body of black ginger. It is also assumed that the components responsible for the distinctive flavor of black ginger will remain in its plant body without transferring into the oil or fat.

Subsequently, after extraction has been performed, the oil or fat extract as obtained by the extraction is optionally freed of insoluble solids by filtration or centrifugation.

Alternatively, the production of an oil or fat extract comprises contacting a plant body of black ginger with water, a hydrophilic solvent or a mixture thereof and extracting one or more members selected from the following eleven methoxyflavones, i.e., 5,7,3′,4′-tetramethoxyflavone, 3,5,7,3′,4′-pentamethoxyflavone, 5,7-dimethoxyflavone, 5,7,4′-trimethoxyflavone, 3,5,7-trimethoxyflavone, 3,5,7,4′-tetramethoxyflavone, 5-hydroxy-3,7,3′,4′-tetramethoxyflavone, 5-hydroxy-7-methoxyflavone, 5-hydroxy-7,4′-dimethoxyflavone, 5-hydroxy-3,7-dimethoxyflavone, and 5-hydroxy-3,7,4′-trimethoxyflavone, followed by bringing an intermediate extract as obtained by the extraction into contact with an oil or fat and extracting said one or more methoxyflavones. A typical example of this method is described below.

First, a plant body of black ginger is provided as described above. Subsequently, the plant body or part of it is brought into contact with water, a hydrophilic solvent or a mixture thereof and subjected to extraction. The conditions for extraction are not particularly limited as long as they are capable of extracting methoxyflavones. Typical extraction temperatures are room temperature to reflux temperature, 40° C. to reflux temperature, 50° C. to reflux temperature, or at reflux temperature, preferably 50° C. to reflux temperature or at reflux temperature. The extraction time is typically one minute to a day, 10 minutes to 10 hours, or 15 minutes to 5 hours. And the volume of the water, hydrophilic solvent or mixture thereof to be used is typically 0.1 to 30 times or 0.5 to 15 times the weight of black ginger. The hydrophilic solvent to be used is preferably a C₁₋₃ alcohol and/or acetone, more preferably ethanol. For example, 50-100 v/v % ethanol may be used as an extraction solvent. The intermediate extract obtained by this extraction step is subjected to the next step of extraction with an oil or fat.

In the oil or fat extraction step, the intermediate extract is contacted with an oil or fat to perform extraction. The conditions for extraction are not particularly limited as long as they are capable of extracting methoxyflavones. The extraction temperature is not particularly limited and the process may be performed at, for example, 5° C. and above, or 10° C. and above, or 20° C. and above, or 30° C. and above, or 40° C. and above, or 50° C. and above. The extraction temperature has no particular upper limit and may be of any value that does not exceed the reflux temperature of the water, hydrophilic solvent or mixture thereof. The extraction time is typically one minute to a day, 10 minutes to 10 hours, or 15 minutes to 5 hours. And the volume of the oil or fat to be used is typically 0.01 to 30 times or 0.5 to 15 times the weight of black ginger. Examples of the oil or fat to be used are as already described above.

Further, depending on the case, the water, hydrophilic solvent or mixture thereof is evaporated from the intermediate extract before it is contacted with the oil or fat and/or while they are kept in contact with each other. The evaporation may be performed at ordinary or reduced pressure. In the case of performing such positive evaporation, the extraction time is not very important. Presumably, as evaporation proceeds and the amount of the water, hydrophilic solvent or mixture thereof decreases, methoxyflavones will transfer into the oil or fat, occasionally together with the hydrophilic solvent and the like.

Although not wishing to be bound by any theory, the present inventor assumes that in the process of oil or fat extraction, methoxyflavones transfer into the oil or fat whereas the components that are responsible for the black purple color of black ginger will not.

Subsequently, after extraction has been performed, the oil or fat containing extract obtained by the extraction is optionally freed of insoluble solids by filtration or centrifugation. This also applies to the intermediate extract.

According to the two methods described above, an oil or fat containing extract can be obtained. This extract may be used without further purification but, if necessary, it may be purified. For example, the oil or fat containing extract may be subjected to a step of further extraction to remove the oil or fat. Specifically, the oil or fat containing extract is contacted with water, a hydrophilic solvent or a mixture thereof to extract one or more of the methoxyflavones described above. In the process, if there is a need, a solvent of low polarity such as a C₁₋₈ hydrocarbon like n-hexane may be added to the oil or fat containing extract.

Examples of the hydrophilic solvent or its mixture with water to be used are as described above. The extraction temperature is not particularly limited and the process may be performed at, for example, 5° C. and above, or 10° C. and above, or 20° C. and above, or 30° C. and above, or 40° C. and above, or 50° C. and above. The extraction temperature has no particular upper limit and may be of any value that does not exceed the reflux temperature of the water, hydrophilic solvent or mixture thereof. The extraction time is typically one minute to a day, 10 minutes to 10 hours, or 15 minutes to 5 hours. And the volume of the water, hydrophilic solvent or mixture thereof to be used is typically 0.01 to 30 times or 0.5 to 15 times the weight of the oil or fat extract.

Further in addition, during the process of extracting methoxyflavones, there is obtained a two-phase mixture of an oil or fat phase derived from the oil or fat containing extract and a phase derived from the water, hydrophilic solvent or mixture thereof, and this mixture is subjected to liquid-liquid separation. As a result, the oil or fat phase can be separated from the phase of the water, hydrophilic solvent or mixture thereof (which is an extract containing methoxyflavones and the solvent). For liquid-liquid separation, the two-phase mixture may, for example, be simply left to stand or it may be subjected to centrifugation. Subsequently, the separated extract is recovered.

The separated extract is in the form of a liquid that contains at least one member selected from the following eleven methoxyflavones, i.e., 5,7,3′,4′-tetramethoxyflavone, 3,5,7,3′,4′-pentamethoxyflavone, 5,7-dimethoxyflavone, 5,7,4′-trimethoxyflavone, 3,5,7-trimethoxyflavone, 3,5,7,4′-tetramethoxyflavone, 5-hydroxy-3,7,3′,4′-tetramethoxyflavone, 5-hydroxy-7-methoxyflavone, 5-hydroxy-7,4′-dimethoxyflavone, 5-hydroxy-3,7-dimethoxyflavone, and 5-hydroxy-3,7,4′-trimethoxyflavone, and which also contains the solvent. This liquid may be directly put to use or, alternatively, the solvent (water, hydrophilic solvent or mixture thereof) may be removed to yield an extract in powder form that contains methoxyflavones. The method for removing the solvent is not particularly limited and examples include distillation under ordinary or reduced pressure, freeze-drying, and so on.

In the extract thus freed of oil or fat, methoxyflavones which are characteristic of black ginger are contained at comparatively high concentrations. If necessary, this extract may also be subjected to further purification.

The oil or fat extracts obtained in accordance with the above-described methods (including the oil or fat containing extract, and the extract freed of oil or fat) permit an optional addition of “Other components” described above, including antioxidants.

(Applications)

The oil or fat extracts of the present invention can be used as foods or beverages (e.g. functional foods, health supplements, foods with nutrient function claims, foods for special dietary uses, foods for specified health uses, nutritional supplements, foods for medical diet, health foods, dietary supplements, etc.), pharmaceuticals or cosmetics, or as starting materials therefor. The foods or beverages and pharmaceuticals may be pet foods, animal feeds, etc. that are processed as feeds for pets, as well as veterinary pharmaceuticals.

The foods or beverages, pharmaceuticals and cosmetics can be used to obtain a variety of physiological actions, such as anti-oxidizing, anti-obesity, anti-allergic, anti-inflammatory, anti-ED, and blood stream improving actions, that are considered to be effectively exhibited by methoxyflavones.

The total amount of methoxyflavones to be contained in foods or beverages, pharmaceuticals and cosmetics varies with their forms and uses but it is preferably about 0.0001 to about 10 wt %, with the range about 0.05 to about 5 wt % being particularly preferred.

The form of foods or beverages that contain the oil or fat extracts of the present invention is not particularly limited and examples include soft drinks (e.g. sports drinks, carbonated drinks, fruit juice containing drinks), confectionery (e.g. cakes, biscuits, breads, candies), noodles (e.g. udon, soba, ramen, pasta), miso, soy sauce, vinegar, salad oil, sesame oil, soy milk, and cow milk. Other possible forms include tablets, granules, powders, capsules (including soft capsules), etc. These can be produced by known methods using the oil or fat extracts according to the present invention and other materials that are commonly used, such as excipients and diluents.

The form of pharmaceuticals that contain the oil or fat extracts of the present invention is not particularly limited and examples include preparations for external application (e.g. lotions, emulsions, patches, ointments) and oral preparations (tablets, granules, powders, capsules (including soft capsules), solutions, suspensions). These can be produced by known methods using the oil or fat extracts according to the present invention and other materials that are commonly used, such as excipients and diluents.

The form of cosmetics that contain the oil or fat extracts of the present invention is not particularly limited and examples include toilet waters, jells, lotions, creams, face masks, milk emulsions, foundations, lipsticks, powder rouges, facial washes, and hair tonics. These can also be produced by known methods using the oil or fat extracts according to the present invention and other materials that are commonly used, such as excipients and diluents.

The present inventor has also found that the oil or fat extracts of the present invention are effective as a NADPH oxidase (NOX) inhibitor. NOX, typically occurring in basophils, is an enzyme known to generate O²⁻. Inhibiting NOX leads to preventing or treating NOX-associated diseases. Hence, the oil or fat extracts of the present invention can also be used to prevent or treat NOX-associated diseases. Such diseases include: allergic diseases such as atopic dermatitis, allergic rhinitis (pollinosis), allergic conjunctivitis, allergic gastroenteritis, bronchial asthma, infantile asthma, food allergy, drug allergy and hives; Parkinson's disease; cerebral infarction; cataract; epilepsy; spinal cord injury; arteriosclerosis; retinopathy of prematurity; renal disorder; peptic ulcer; pancreatitis; ulcerative colitis; myocardial infarction; adult respiratory distress syndrome; pulmonary emphysema; collagen diseases such as chronic rheumatoid arthritis; angiitis; edema; complications of diabetes; ultraviolet disorders; altitude sickness; porphyria; burns; frostbite; contact dermatitis; shock; failure of multiple organs; DIC; cancer; aging; fatigue; sarcopenia (progressive decline in skeletal muscle mass); mitochondrial dysfunction; dementia; Alzheimer's disease.

(Numerical Ranges)

For the purpose of clarity, it should be noted that whenever a numerical range is defined herein by its lower and upper limits and expressed as “lower limit to upper limit,” both the lower and upper limits are included. For example, the range expressed as “1 to 2” includes 1 and 2.

### EXAMPLES

### Example 1

### Isolation and Purification of Methoxyflavones

To 150 g of black ginger, 1500 ml of a 50% aqueous ethanol solution was added and the mixture was heated under reflux for 2 hours to perform extraction. After cooling, the resulting liquid extract was filtered, concentrated under reduced pressure and freeze-dried to yield 25.7 g of a black ginger extract. A 9 g portion of the resulting extract was subjected to column chromatography using Dia ion HP20 (product of Mitsubishi Chemical Corporation) and fractionated into four fractions (a portion eluted with 30% ethanol; a portion eluted with 50% ethanol; a portion eluted with 70% ethanol; and a portion eluted with 100% ethanol). The portion eluted with 50% ethanol was subjected to high-speed liquid chromatography to isolate 5,7,3′,4′-tetramethoxyflavone (64 mg), 3,5,7,3′,4′-pentamethoxyflavone (464 mg), 5,7-dimethoxyflavone (145 mg), 5,7,4′-trimethoxyflavone (188 mg), 3,5,7-trimethoxyflavone (35 mg), and 3,5,7,4′-tetramethoxyflavone (96 mg). Subsequently, the portion eluted with 100% ethanol was also subjected to the same procedures of separation and purification by liquid chromatography to isolate 5-hydroxy-3,7,3′,4′-tetramethoxyflavone (15 mg), 5-hydroxy-7-methoxyflavone (84 mg), 5-hydroxy-7,4′-dimethoxyflavone (56 mg), 5-hydroxy-3,7-dimethoxyflavone (100 mg), and 5-hydroxy-3,7,4′-trimethoxyflavone (110 mg). The isolated compounds were identified by comparing their spectrum data with the various spectrum data presented in a document (“The Structures of Components in A Plant of the Zingiberaceae Family, Kaempferia parviflora, as well as Their α-Glucosidase Inhibitory Activities and Antimutagenicities” which is a doctor's thesis of Mr. Toshiaki Azuma, Graduate School of Human Life Science, Osaka City University).

### Example 2

### Production of Oil or Fat Extracts

Two samples of black ginger (3 g and 15 g) were each mixed with 30 mL of olive oil and subjected to extraction at 120° C. for 30 minutes; thereafter, the mixtures were cooled and filtered to yield two pale yellow oil or fat extracts of black ginger. Under the analytical conditions set forth below, the two pale yellow oil or fat extracts were determined for their total contents of eleven methoxyflavones (compounds 1 to 11 listed in Table 1); the respective values were 6.2 mg/mL (from the black ginger weighing 3 g) and 22.4 mg/mL (from the black ginger weighing 15 g). Each of the extracts contained all of compounds 1 to 11 listed in Table 1.

(Analysis and Quantification of Methoxyflavones)

To 1.0 mL of a black ginger's oil or fat extract, 1.0 mL of n-hexane was added for dilution and, thereafter, three extractions of methoxyflavones were conducted with 2.0 mL of an 80% aqueous methanol solution. In each run, the resulting 80% liquid methanol extract was passed through Mega Bond Elute C18 (product of Agilent Technologies Japan, Ltd.), followed by passing 2.0 mL of 80% methanol with a view to washing out the methoxyflavones adsorbed on Mega Bond Elute C18. The resulting liquids were combined and diluted to a final volume of 10 mL for use as a sample for analysis by HPLC.

(Conditions for HPLC Analysis)

Column: Develosil C30 UG5 (4.6×150 mm, 5 μm; product of Nomura Chemical Co., Ltd.)

Detection: 280 nm (200-600 nm for PDA detection)

Column temperature: 40° C.

Mobile phase A: 0.05% trifluoroacetic acid in aq. sol.

Mobile phase B: 0.05% trifluoroacetic acid solution in 90% acetonitrile in aq. sol.

Gradient: Mobile phase B 50%→50%→70%→70% (0 min→7.5 min→20 min→25 min)

Flow rate: 1.0 mL/min

Sample injection: 10 μL

### Example 3

To 50 g of black ginger, 500 mL of ethanol was added and the mixture was heated under reflux for an hour to perform extraction. The resulting liquid was cooled and thereafter filtered by suction; to the resulting liquid extract, 15 mL of middle-chain fatty acid triglyceride was added and after distilling off ethanol by concentrating under reduced pressure, another filtration was performed by suction with a view to removing the insoluble matter, whereby an oil or fat extract of black ginger was obtained. The same procedures of ethanol extraction were performed to give two more liquid extracts, to which 15 ml of olive oil or a liquid mixture of a middle-chain fatty acid triglyceride (Nisshin MCT Oil; product of The Nisshin Oillio Group, Ltd.) and olive oil (mixed at a ratio of 1:1) was added and after distilling off ethanol by concentrating under reduced pressure, the precipitating insoluble matter was removed through filtration by suction, yielding two additional oil or fat extracts of black ginger. The thus obtained three oil or fat extracts of black ginger were analyzed for the total contents of eleven methoxyflavones as described in Example 2; the respective values were 34.7 mg/mL (as extracted with middle-chain fatty acid triglyceride), 4.5 mg/mL (olive oil), and 6.7 mg/mL (liquid mixture of middle-chain fatty acid triglyceride and olive oil). From these data, it became clear that methoxyflavones have high solubility in the middle-chain fatty acid triglyceride which therefore is suitable as a solvent. It should be noted that each of these extracts contained all of compounds 1 to 11 listed in Table 1.

### Example 4

To 30 g of black ginger, 30 mL of a liquid mixture of middle-chain fatty acid triglyceride and olive oil (mixed at a ratio of 1:1) was added to make three suspensions; after performing extraction at 100° C., 120° C. and 150° C. (each for a period of 30 min), filtration was conducted to yield three oil or fat extracts. The total content of eleven methoxyflavones in each extract was measured by the same method as in Example 2. The respective values were 6.8 mg/mL (100° C.), 22.6 mg/mL (120° C.), and 24.4 mg/mL (150° C.), demonstrating that the higher the temperature, the more efficient was the extraction. It should be noted that each of these extracts described above contained all of compounds 1 to 11 listed in Table 1.

### Example 5

To check for the difference that might be caused to the composition of oil or fat extracts from different lots of black ginger, two black ginger samples each weighing 200 g were provided and to each sample, 1000 mL of ethanol was added and the mixture was heated under reflux for an hour to perform extraction. The resulting liquid was cooled and thereafter filtered by suction to be separated into the residue and the liquid extract. To the residue, 1000 mL of ethanol was added again and the mixture was heated under reflux for an hour to perform extraction; after filtration, the resulting liquid extract was combined with the previously obtained liquid extract. Subsequently, 100 mL of middle-chain fatty acid triglyceride was added to the combined liquid extracts and after distilling off ethanol by concentrating under reduced pressure, the precipitating insoluble matter was removed through filtration by suction to yield two oil or fat extracts of black ginger. When the contents of methoxyflavones in these extracts were analyzed as in Example 2, the total amount of methoxyflavones was 90.4 mg/mL (in the extract which is hereinafter referred to as Extract A) and 54.9 mg/mL (in the extract which is hereinafter referred to as Extract B). Using olive oil, the total amount of methoxyflavones in each of these two extracts was adjusted to 5 mg/ml; the thus obtained two solutions were measured for absorbance at 660 nm, which was 0.036 (for Extract A) and 0.030 (for Extract B), with methanol being used as a blank. It should be noted that each of these extracts contained all of compounds 1 to 11 listed in Table 1.

### Comparative Example 1

To 200 g of dried black ginger, 1000 mL of a 50% aqueous ethanol solution was added and the mixture was heated under reflux for an hour to perform extraction. The resulting liquid was cooled and thereafter filtered by suction to be separated into the residue and the liquid extract. To the residue, 1000 mL of a 50% aqueous ethanol solution was added again and the mixture was heated under reflux for an hour to perform extraction; after filtration, the resulting liquid extract was combined with the previously obtained liquid extract. After cooling to room temperature, the combined liquid extracts were concentrated under reduced pressure and then freeze-dried to yield black ginger's ethanol extract No. 1 in an amount of 49 g (yield: 24.5%). To check for the difference that might be caused to the composition of extracts from different lots of black ginger, the same procedure as described above was taken to yield black ginger's ethanol extract No. 2 in an amount of 23 g (yield: 15.2%). Subsequently, the total contents of eleven methoxyflavones in these extracts were analyzed by the same method as in Example 2; the respective values were 264 mg/g and 267 mg/g. Using methanol, the total amount of methoxyflavones in black ginger's ethanol extract No. 1 was adjusted to 5 mg/ml; the thus obtained solution was measured for absorbance at 660 nm, which was 0.95 (the blank was methanol). This value was far greater than those obtained from the extracts of the present invention in Example 5. It should be noted that each of these extracts contained all of compounds 1 to 11 listed in Table 1.

### Comparative Example 2

A commercial black ginger extract (trade name: Black Ginger Extract; product of Maruzen Pharmaceuticals Co., Ltd.) was treated with a 50% aqueous methanol solution so that its total amount of methoxyflavones was adjusted to 5 mg/ml; the thus obtained solution was measured for absorbance at 660 nm, which was 1.960 (the blank was methanol).

### Example 6

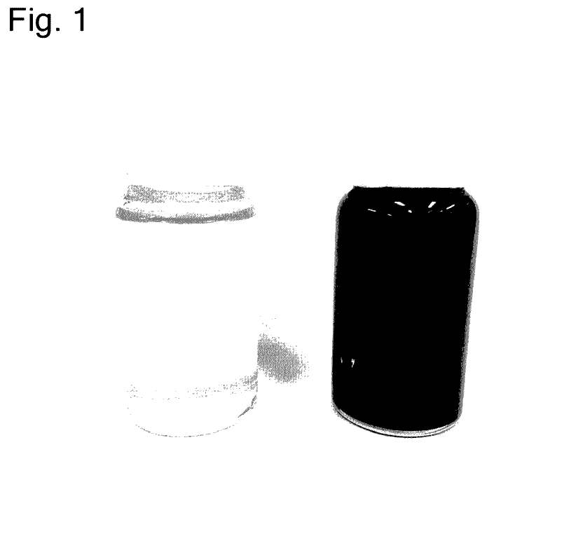

Photos were taken of an oil or fat extract (A) prepared from black ginger substantially in accordance with the method described in Example 4 (extracted with olive oil at 120° C.) and a hydrous alcohol extract (B) prepared from black ginger substantially in accordance with the method described in Comparative Example 1. The oil or fat extract (A) had its concentration adjusted with olive oil so that the total methoxyflavone content would be 5 mg/ml. Similarly, the hydrous alcohol extract (B) had its concentration adjusted with a 50% aqueous ethanol solution so that the total methoxyflavone content would be 5 mg/ml. The result is shown in FIG. 1; the left photo shows the oil or fat extract (A) and the right photo shows the extract (B). Obviously, the oil or fat extract of the present invention was less colored than the hydrous alcohol extract.

### Example 7

To a 10-g portion of each of the two black ginger's oil or fat extracts A and B as obtained in Example 5, 10 mL of n-hexane was added and the mixture was contacted with an 80% aqueous ethanol solution (20 mL), whereupon methoxyflavones were transferred and dissolved into the aqueous ethanol solution. The thus obtained mixture was left to stand until the phase of the aqueous ethanol solution separated from the oil or fat phase. The separated aqueous ethanol solution phase was recovered and the solvent was distilled off under reduced pressure, whereupon a yellow powder containing methoxyflavones was obtained from Extract A in an amount of 1.03 g and from Extract B in an amount of 0.68 g. It should be noted that each of these powders contained all of compounds 1 to 11 listed in Table 1.

### Example 8

### Preparing Samples for NOX Inhibitory Activity Measurement

To each of black ginger samples weighing 10 g, 20 g, 30 g and 40 g, 10 volumes of ethanol were added and the mixture was heated under reflux for an hour to perform extraction. The resulting liquid was cooled and then filtered by suction; to the resulting liquid extract, 15 mL of middle-chain fatty acid triglyceride was added and ethanol was distilled off by concentrating under reduced pressure; thereafter, with a view to removing the insoluble matter, filtration by suction was performed again, yielding four black ginger's oil or fat extracts. The thus obtained four black ginger's oil or fat extracts were analyzed for the total contents of eleven methoxyflavones substantially in accordance with Example 2; the respective values were 23.9 mg/mL, 46.3 mg/mL, 69.4 mg/mL, and 78.1 mg/mL. When the oil or fat extracts containing the methoxyflavones at a concentration of 46.3 mg/mL or more were left to stand at room temperature, methoxyflavones were found to precipitate. It should be noted that each of these extracts contained all of compounds 1 to 11 listed in Table 1.

### Example 9

### NOX Inhibitory Activity of Black Ginger's Oil or Fat Extracts

Preparing Differentiated HL-60 Cells:

Human myeloid leukemia cell HL-60 repeats proliferation in an undifferentiated state but upon addition of DMSO (dimethyl sulfoxide), retinoic acid or the like, it is known to differentiate into mature granulocytes and lose the ability to proliferate, as accompanied by intracellular expression of NOX (NADPH oxidase) which also serves as an index for differentiation; the expressed NOX can be utilized as an enzyme source for evaluating NOX inhibitory activity.

To induce differentiation into NOX expressing granulocytes, undifferentiated HL-60 cells cultured in a 10% FBS supplemented RPMI 1640 medium were suspended in a 1% DMSO containing, 10% FBS supplemented RPM I1640 medium to give a density of 5×10⁵ cells/ml, and the suspension was distributed among Petri dishes (i.d. 10 cm) in 15 ml portions and cultured in a CO₂ incubator (37° C.) for three days; thereafter, 10 ml of a 1% DMSO containing, 10% FBS supplemented RPM I1640 medium was added into each of the Petri dishes and culture was performed for an additional three days, thus yielding differentiated HL-60 cells in which NOX was expressed. As described below, the differentiated HL-60 cells, either as a homogenate or in a viable state, were subjected to NOX activity measurement.

NOX Activity Measurement in a Cell-Free System Using a Homogenate:

HL-60 cells differentiated by DMSO treatment were collected by centrifugation and, after being washed once with PBS (phosphate buffered physiological saline), suspended in a homogenizing buffer (8 mM phosphate buffered solution containing 131 mM NaCl and 340 mM sucrose; pH 7.0) to give a density of 1×10⁸ cells/ml. After being cooled with ice, the suspension was treated with a sonicator (Bioruptor UCD-250 HSA; product of Cosmo Bio Co., Ltd.) by repeating three cycles of a process under the condition of 4° C. or below that consisted of 20-sec disrupting at maximum power and 30-sec interval cooling, whereupon a cell homogenate was obtained. The homogenate was centrifuged at 1000 g for 4 minutes to remove debris; to the resulting supernatant, nine volumes of a reaction buffer (65 mM phosphate buffered solution containing 1 mM EGTA, 10 μM FAD and 170 mM sucrose; pH 7.0) were added to prepare a homogenate's supernatant for NOX measurement (equivalent to 1×10⁷ cells/nil).

For reaction with NOX, 50 μl of the above-described cell homogenate was poured into each well of a 96-well microplate and after adding 25 μl of a 0.5 mM SDS solution as a NOX activator and 25 μl of a 0.4 mM NADPH solution as a substrate, the reaction was carried out at 25° C. for 30-90 minutes. The NOX activity was determined by measuring the rate of NADPH consumption through fluorometry (Ex: 355 nm/Em: 460 nm).

To measure the NOX inhibitory activity of a test sample, a DMSO solution of the sample (usually 10 mM for a reagent) was prepared, from which 3-fold serial dilutions were prepared by adding DMSO; the dilutions were each added in a volume of 1 μl/well to the above-described reaction solution to thereby carry out an enzymatic reaction; the resulting inhibitory activity was indicated in terms of IC₅₀ value (in μM, or μg/ml for extract).

NOX Activity Measurement Using Viable, Differentiated HL-60 Cells:

HL-60 cells differentiated by DMSO treatment were collected by centrifugation and suspended in a FBS- and Phenol Red-free D-MEM medium to give a density of 5×10⁶ cells/ml. For reaction with NOX, 25 μl of the above-described cell suspension was poured into each well of a 96-well microplate and, furthermore, 0.8 mg/ml of a WST-1 solution as prepared using the above-described D-MEM and a test sample dissolving solution as prepared at a predetermined concentration (i.e., a DMSO solution of the sample (usually 10 mM for a reagent) was prepared, from which 3-fold serial dilutions were prepared using DMSO and added to the above-described D-MEM to give a concentration of 1 v/v % or below, thereby preparing the test sample dissolving solution of interest) were each added in a 25 μl portion, followed by stirring of the mixture; thereafter, 25 μl of 4 μM PMA (Phorbol 12-Myristate 13-acetate in a final concentration of 1 μM) as dissolved in D-MEM was added to activate NOX; reaction was carried out at 37° C. for 45 minutes, whereupon superoxide as the NOX enzymatic product reacted with WST-1 in the reaction solution to generate yellow formazan and its quantity was measured in terms of absorbance at 450 nm. It should be noted that in this system of NOX activity measurement, NOX would not be activated unless PMA was added.

The resulting inhibitory activity was indicated in terms of IC₅₀ value (in μM, or μg/ml for extract).

Results:

In accordance with the above-described procedures, black ginger's oil or fat extract No. 1 with a total methoxyflavone content of 22.4 mg/ml (as obtained in Example 2), black ginger's oil or fat extract No. 2 with a total methoxyflavone content of 69.4 mg/ml (as obtained in Example 8) and an ethanol extract (black ginger's ethanol extract Nos. 1 and 2 as obtained in Comparative Example 1) were measured for NOX inhibitory activity (NOX inhibitory activity measurement using viable, differentiated HL-60 cells). To check for the difference in activity between lots of black ginger, investigation was made using two types of black ginger. The oil or fat extracts as just prepared would defy activity measurement, so they were degreased; specifically, 0.5 mL of an oil or fat extract was diluted with the same quantity (0.5 mL) of added n-hexane and, thereafter, methoxyflavones were extracted three times with 0.5 mL of an 80% aqueous methanol solution; the resulting liquid extract was adsorbed on Sep-Pak PLUS C8 125 Å Cartridges (product of Waters), through which 3.0 mL of 80% methanol was passed to remove the oil content. Thereafter, Sep-Pak PLUS C8 125 Å Cartridges was washed with a solvent and the resulting liquid was concentrated under reduced pressure and freeze-dried to prepare samples for evaluation. The measured IC₅₀ values are shown in Table 2 below.

The IC₅₀ values shown below represent the NOX inhibitory activity based on the total methoxyflavone content. Upon comparison of those values, the oil or fat extracts were both shown to have higher action (lower IC₅₀) than the ethanol extracts. This suggested that methoxyflavones having higher inhibitory action on NOX were extracted efficiently in the present invention by using oils or fats as solvents.

TABLE 2

| | NOX IC₅₀ (μg/ml) |
|---|---|
| | Calculated for the total |
| Extract | methoxyflavone content |
| black ginger's ethanol extract No. 1 | 11.7 |
| black ginger's oil or fat extract No. 1 | 9.2 |
| black ginger's ethanol extract No. 2 | 7.7 |
| black ginger's oil or fat extract No. 2 | 3.1 |

### Example 10

### Relation Between Extraction Method and Composition

In view of the results of Example 9, compositional comparison was made between two types of extracts from black ginger, one obtained by extraction with oil or fat and another obtained by extraction with a hydrophilic solvent. Specifically, extraction with oil or fat was conducted substantially in accordance with Examples 2 to 6 and 8 (extraction with oil or fat only, or extraction with ethanol followed by extraction with oil or fat) and extraction with ethanol was conducted substantially in accordance with Comparative Example 1. In the oil or fat extraction, olive oil or a mixture of olive oil with middle-chain fatty acid glyceride was used (in Example 10, a middle-chain fatty acid triglyceride was used, as sometimes denoted by MCT). The resulting extracts were analyzed by HPLC based on the method described in Example 2 and the obtained peak area data are shown below. In the following Tables, oil or fat is sometimes simply referred to as Oil for the sake of convenience. The compound numbers for methoxyflavones correspond to the compound numbers listed in Table 1.

TABLE 3A

| | Extraction method | | | | | | | | | |
|---|---|---|---|---|---|---|---|---|---|---|
| Methoxyflavones | EtOH [symbol] Oil | | EtOH | | Oil | | | | | |
| 1 | 38.2 | 6.5 | 11.2 | 28.9 | 19.1 | 23.7 | 11.8 | 10.2 | 7.4 | 7.2 |
| 2 | 207.2 | 36.2 | 57.8 | 134.2 | 95.4 | 119.5 | 72.3 | 62.9 | 48.1 | 46.7 |
| 3 | 633.8 | 129.1 | 69.2 | 145.0 | 103.1 | 127.5 | 137.1 | 119.7 | 67.7 | 65.7 |
| 4 | 391.8 | 71.9 | 82.5 | 187.6 | 132.0 | 164.0 | 108.6 | 94.4 | 68.4 | 66.3 |
| 5, 6 | 289.1 | 56.7 | 49.5 | 109.7 | 81.3 | 106.2 | 82.0 | 71.6 | 46.6 | 45.1 |
| 7 | 16.9 | 3.4 | 6.6 | 13.9 | 9.0 | 10.4 | 7.3 | 6.4 | 4.6 | 4.3 |
| 8 | 42.9 | 8.5 | 14.3 | 35.0 | 15.9 | 17.2 | 14.8 | 13.1 | 8.4 | 7.9 |
| 9 | 22.9 | 4.2 | 8.4 | 20.8 | 10.6 | 12.1 | 9.9 | 8.7 | 6.1 | 5.8 |
| 10 | 48.3 | 9.9 | 19.1 | 47.0 | 17.3 | 19.7 | 18.7 | 16.0 | 10.0 | 9.4 |
| 11 | 41.9 | 8.2 | 19.7 | 45.6 | 19.3 | 22.1 | 21.2 | 17.6 | 12.2 | 11.5 |
| A | 1271.0 | 243.7 | 220.8 | 495.7 | 349.6 | 434.6 | 329.8 | 287.2 | 191.6 | 185.9 |
| A + B | 1732.9 | 334.5 | 338.5 | 767.6 | 503.0 | 622.3 | 483.7 | 420.5 | 279.4 | 269.8 |
| A/(A + B) | 0.73 | 0.73 | 0.65 | 0.65 | 0.69 | 0.70 | 0.68 | 0.68 | 0.69 | 0.69 |

TABLE 3B

| | Extraction method | | | | | | | | |
|---|---|---|---|---|---|---|---|---|---|
| | Oil (containing MCT) | | | | | | | | |
| Methoxyflavones | 33% | 50% | 67% | 33% | 50% | 67% | 33% | 50% | 67% |
| 1 | 6.8 | 4.2 | 6.6 | 17.7 | 23.6 | 22.4 | 25.6 | 27.1 | 25.3 |
| 2 | 39.6 | 24.0 | 36.6 | 105.0 | 137.6 | 128.6 | 150.7 | 156.2 | 145.1 |
| 3 | 137.4 | 87.8 | 131.7 | 354.2 | 453.4 | 421.1 | 474.5 | 470.9 | 443.0 |
| 4 | 75.8 | 48.1 | 72.4 | 198.1 | 256.9 | 242.6 | 277.6 | 285.1 | 266.8 |
| 5, 6 | 62.2 | 37.9 | 57.2 | 162.0 | 207.2 | 190.5 | 220.9 | 220.4 | 205.6 |
| 7 | 4.1 | 2.3 | 3.4 | 10.1 | 13.1 | 11.5 | 14.7 | 15.3 | 13.1 |
| 8 | 10.3 | 5.8 | 8.6 | 27.6 | 35.1 | 30.2 | 38.6 | 42.1 | 32.7 |
| 9 | 5.2 | 2.8 | 4.3 | 13.9 | 17.8 | 16.2 | 20.7 | 22.6 | 17.7 |
| 10 | 12.2 | 6.8 | 10.0 | 32.1 | 40.4 | 35.7 | 44.5 | 48.7 | 37.1 |
| 11 | 10.0 | 5.5 | 8.0 | 26.5 | 33.6 | 30.3 | 38.7 | 42.1 | 32.7 |
| A | 259.6 | 164.1 | 247.3 | 675.0 | 871.4 | 814.7 | 928.5 | 939.3 | 880.2 |
| A + B | 363.6 | 225.1 | 338.8 | 947.2 | 1218.7 | 1129.1 | 1306.5 | 1330.4 | 1219.1 |
| A/(A + B) | 0.71 | 0.73 | 0.73 | 0.71 | 0.72 | 0.72 | 0.71 | 0.71 | 0.72 |

*Percentages in the table represent the relative contents of MCT in the oil or fat used in extraction.

As is clear from Tables 3A and 3B, the oil or fat extracts had higher values of A/(A+B), or higher proportions of methoxyflavones of group A with high NOX inhibitory activity, than the ethanol extracts. Such differences in composition can affect the NOX inhibitory activity.

1. An oil or fat extract, as obtained from black ginger, which comprises one or more members selected from the following eleven methoxyflavones, i.e., 5,7,3′,4′-tetramethoxyflavone, 3,5,7,3′,4′-pentamethoxyflavone, 5,7-dimethoxyflavone, 5,7,4′-trimethoxyflavone, 3,5,7-trimethoxyflavone, 3,5,7,4′-tetramethoxyflavone, 5-hydroxy-3,7,3′,4′-tetramethoxyflavone, 5-hydroxy-7-methoxyflavone, 5-hydroxy-7,4′-dimethoxyflavone, 5-hydroxy-3,7-dimethoxyflavone, and 5-hydroxy-3,7,4′-trimethoxyflavone, wherein a solution which is prepared from said extract and in which the total content of said eleven methoxyflavones is 5.0 mg/ml shows an absorbance of 0.10 or less as measured at a wavelength of 660 nm.

2. The extract according to claim 1, wherein the oil or fat is at least one member selected from among middle-chain fatty acid triglycerides, diacylglycerol, sesame salad oil, olive oil, soybean oil, rapeseed oil, corn oil, rice germ oil, sunflower seed oil, Perilla frustescens var. crispa oil, and Perilla frustescens var. frustescens oil.

3. A food or beverage comprising the extract according to claim 1.

4. A cosmetic comprising the extract according to claim 1.

5. A process for producing from black ginger an oil or fat extract which comprises one or more members selected from the following eleven methoxyflavones, i.e., 5,7,3′,4′-tetramethoxyflavone, 3,5,7,3′,4′-pentamethoxyflavone, 5,7-dimethoxyflavone, 5,7,4′-trimethoxyflavone, 3,5,7-trimethoxyflavone, 3,5,7,4′-tetramethoxyflavone, 5-hydroxy-3,7,3′,4′-tetramethoxyflavone, 5-hydroxy-7-methoxyflavone, 5-hydroxy-7,4′-dimethoxyflavone, 5-hydroxy-3,7-dimethoxyflavone, and 5-hydroxy-3,7,4′-trimethoxyflavone, which process comprises contacting a plant body of black ginger with an oil or fat and extracting at least one or more of said methoxyflavones.

6. The process according to claim 5, wherein the oil or fat is at least one member selected from among middle-chain fatty acid triglycerides, diacylglycerol, sesame salad oil, olive oil, soybean oil, rapeseed oil, corn oil, rice germ oil, sunflower seed oil, Perilla frustescens var. crispa oil, and Perilla frustescens var. frustescens oil.

7. The process according to claim 5, wherein the extraction is performed at 50 to 180° C.

8. A process for producing from black ginger an oil or fat extract which comprises one or more members selected from the following eleven methoxyflavones, i.e., 5,7,3′,4′-tetramethoxyflavone, 3,5,7,3′,4′-pentamethoxyflavone, 5,7-dimethoxyflavone, 5,7,4′-trimethoxyflavone, 3,5,7-trimethoxyflavone, 3,5,7,4′-tetramethoxyflavone, 5-hydroxy-3,7,3′,4′-tetramethoxyflavone, 5-hydroxy-7-methoxyflavone, 5-hydroxy-7,4′-dimethoxyflavone, 5-hydroxy-3,7-dimethoxyflavone, and 5-hydroxy-3,7,4′-trimethoxyflavone, which process comprises:
contacting a plant body of black ginger with water, a hydrophilic solvent or a mixture thereof and extracting one or more of said methoxyflavones; and
bringing an intermediate extract as obtained by said extraction into contact with an oil or fat and extracting said one or more methoxyflavones.

9. The process according to claim 8 which further includes the step of evaporating the water, hydrophilic solvent or mixture thereof from the intermediate extract before the intermediate extract is contacted with the oil or fat and/or while they are kept in contact with each other.

10. The process according to claim 8, wherein the oil or fat is at least one member selected from among middle-chain fatty acid triglycerides, diacylglycerol, sesame salad oil, olive oil, soybean oil, rapeseed oil, corn oil, rice germ oil, sunflower seed oil, Perilla frustescens var. crispa oil, and Perilla frustescens var. frustescens oil.

11. The process according to claim 5 which further includes the steps of:
bringing an oil or fat containing extract as obtained by the step of contact with an oil or fat into contact with water, a hydrophilic solvent or a mixture thereof and extracting one or more of said methoxyflavones; and
subjecting to liquid-liquid separation a two-phase mixture as obtained during said extraction of said one or more methoxyflavones, whereby an extract comprising the water, hydrophilic solvent or mixture thereof is obtained that has been separated in the liquid-liquid separation step.

12. The process according to claim 11 which further includes the step of removing the water, hydrophilic solvent or mixture thereof from the extract as obtained through the liquid-liquid separation step.

13. The process according to claim 8, wherein the hydrophilic solvent is a C₁₋₃ alcohol and/or acetone.

14. An oil or fat extract as obtained by the process recited in claim 5, which comprises one or more methoxyflavones selected from among said eleven methoxyflavones.
